(19) United States
(12) Patent Application Publication
(10) Pub. No.: US 2026/0221250 A1
(43) Pub. Date: Jul. 30, 2026

(54) SYSTEM AND METHOD FOR HEALTH PRODUCT AND ROUTINE MANAGEMENT

(71) Applicants: Rachel McCullough, Woodland Hills, CA (US); Lani Llacuna-Morales, Woodland Hills, CA (US)

(72) Inventors: Rachel McCullough, Woodland Hills, CA (US); Lani Llacuna-Morales, Woodland Hills, CA (US)

(21) Appl. No.: 19/040,150

(22) Filed: Jan. 29, 2025

(51) Int. Cl. G16H 20/00; G06V 10/82; G06V 40/10; G16H 10/60

(52) U.S. Cl. CPC G16H 20/00; G06V 10/82; G06V 40/10; G16H 10/60

(57) ABSTRACT

A system and method for assessment and identification of products that may be needed by a specific individual based on certain assessment criteria, such as, for example, an individual’s skin type and other health identifiers and the ability to match products that fit with an individual’s skin and health factors and screen for products and routine combinations that may have adverse effects on an individual’s health.

### BACKGROUND

Consumers today face the unique challenge of information overload that was relatively uncommon just a few decades ago. The marketplace is brimming with products, each promising to be the solution to a specific need or want. This excess of options, instead of facilitating choice, often hinders it, creating a paradox of choice that leaves many individuals feeling overwhelmed rather than empowered.

Moreover, the wealth of products is not accompanied by easily accessible, comprehensive information. While some might argue that consumers have all the tools they need to make informed decisions—brand websites, reviews, and more—these resources are dispersed and can be inconsistent in the information they provide.

For example, essential details like whether a product is suitable for a specific skin type, or whether it may address particular concerns are often buried deep in jargon-filled web pages, if they're disclosed at all.

This situation creates a burdensome requirement for consumers to become quasi-experts in the field. They're expected to synthesize information from multiple sources, sifting through conflicting opinions and ambiguous product specifications to finally arrive at a purchasing decision.

The time and effort required for this process are resources that many people simply do not have. As a result, there's a critical need for a solution that consolidates essential information, simplifies the decision-making process, and removes the requirement for consumers to be experts in the field.

For the above reasons, there is a gap in the market and a need for a simplified way to digest all products based on a specific person’s skin type and an individual’s personal preferences and needs.

### SUMMARY

The present invention is directed to system and method for consolidation of essential product information located in fragmented and distributed systems and repositories and inconsistently structured data types and formats from an endless, growing, and changing number of sources and reconfigure such product information into a single source of truth to provide predictive guidance and recommendations based on external skin care factors.

These and other features, aspects, and advantages of the present invention will become better understood with reference to the following description and appended claims.

### BRIEF DESCRIPTION OF DRAWINGS

FIG. 1 shows flow diagram of the system architecture and components thereof.

### DETAILED DESCRIPTION OF THE INVENTION

The detailed description set forth below in connection with the appended drawings is intended as a description of presently-preferred embodiments of the invention and is not intended to represent the only forms in which the present invention may be constructed or utilized. The description sets forth the functions and the sequence of steps for constructing and operating the invention in connection with the illustrated embodiments. It is to be understood, however, that the same or equivalent functions and sequences may be accomplished by different embodiments that are also intended to be encompassed within the spirit and scope of the invention.

The present invention is directed to a system and method for skin care routine management that overcomes the issues of sifting through an endless patchwork of resources, whether verified or not, to easily manage a skin care or health routine in a manageable manner.

The present invention solves the issues relating to the need for development of customized skincare routines, coupled with the provision of real-time feedback regarding how a potential new product integrates into an individual's existing skincare regimen. The system enables users to quickly discern which products are compatible with their skin types and can effectively target their specific concerns.

Some embodiments of the present invention may include features, such as, prediction modules that may predict the type of products that may be needed by a specific individual based on certain assessment criteria, such as, for example, an individual’s skin type and other health identifiers. In certain embodiments of the present invention there may be an assessment module for the prediction, learning, and analysis of end user needs that may be based on a plurality of data points relating to the individual end user of the present invention as well as external factors such as location, environment, climate as well as collective societal data that may include training data from mass data sets to classify certain issues that may arise relating to skin care and health of humans.

In certain embodiments, the present invention may commence a user experience with a comprehensive skin quiz designed to establish parameters for filtering search results, thereby mitigating the overwhelming process of finding appropriate skincare products. These filters may span various factors including, for example, age, location, sun exposure, among other personal and external or environmental factors, which may, in some embodiments, factor into an analysis regarding how certain products may affect a person’s skin or health. Certain skin types and other options may range from, for example, dry, normal, or oily, wherein there may be a myriad of skin concerns such as breakouts, dullness, dark spots, and fine lines among others depending on the situation. In some embodiments, the present invention may also take into account product or brand preferences such as, for example, cruelty-free, gluten-free, vegan, and eco-friendly brands and ingredients.

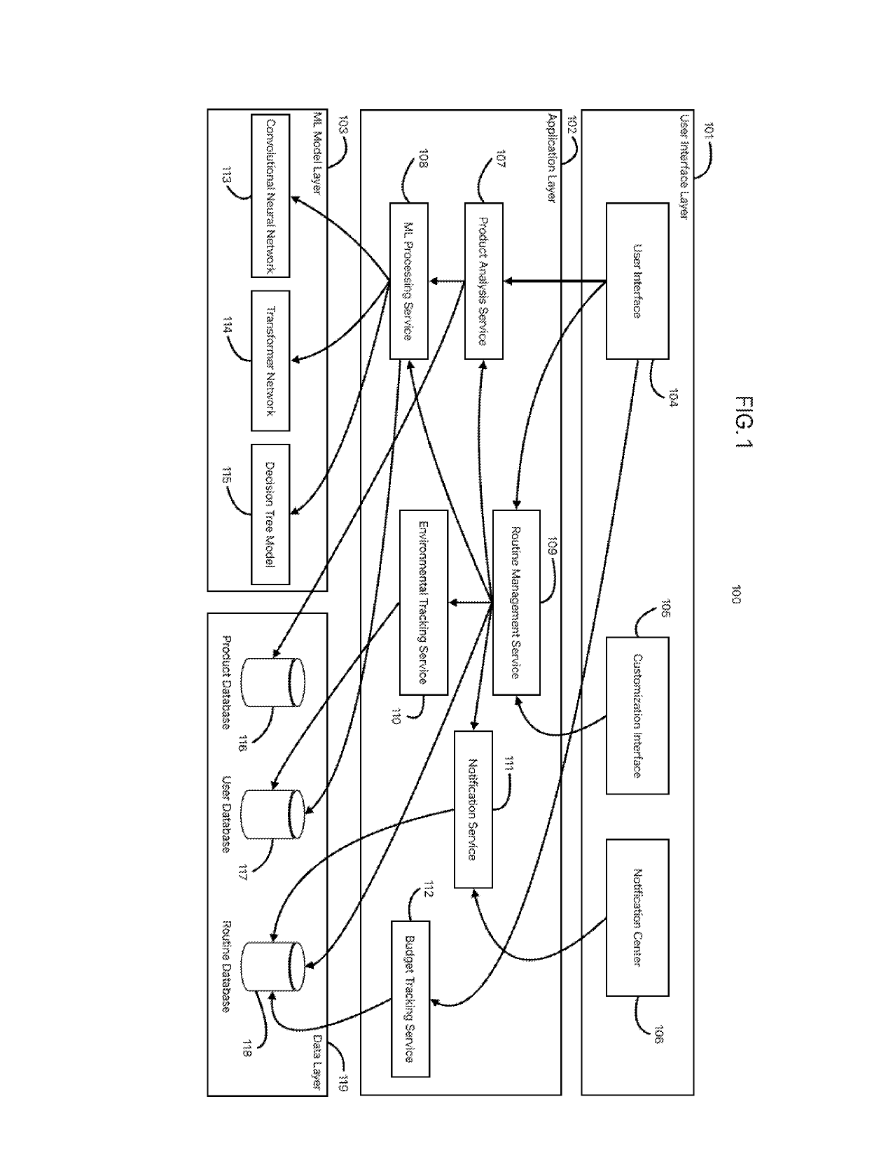

In some embodiments, referring to FIG. 1, a personalized skincare routine management system 100 may comprise a User Interface Layer 101, an Application Layer 102, an ML Model Layer 103, and a Data Layer 119. These layers may cooperate together to provide personalized skincare recommendations and routine management functionalities.

The User Interface Layer 101 may include a User Interface 104, a Customization Interface 105, and a Notification Center 106. In some embodiments, the User Interface 104 may be implemented as a graphical user interface accessible through various devices such as mobile devices, web browsers, or dedicated applications. The User Interface 104 may, for example, provide input mechanisms for receiving user information related to skin type, concerns, preferences, and environmental factors. In certain embodiments, the Customization Interface 105 may enable users to modify their skincare routines, for example, by providing capabilities for adjusting product usage times, adding or removing products, and setting product preferences. The Notification Center 106 may present various system-generated alerts, reminders, and feedback to users regarding their skincare routine.

In some embodiments, the Application Layer 102 may include a Product Analysis Service 107, an ML Processing Service 108, a Routine Management Service 109, an Environmental Tracking Service 110, a Notification Service 111, and a Budget Tracking Service 112. The Product Analysis Service 107 may, for example, analyze skincare product compatibility and interactions by utilizing ingredient databases and user-specific factors. The ML Processing Service 108 may orchestrate the machine learning (referenced as ML herein) models and process input data, and may, in some implementations, handle the routing of different types of data to appropriate ML models. The Routine Management Service 109 may serve as a central coordinator for generating and maintaining personalized skincare routines and may, for example, incorporate user feedback and environmental factors to optimize routines over time.

The ML Model Layer 103 may comprise a Convolutional Neural Network 113, a Transformer Network 114, and a Decision Tree Model 115. In some embodiments, the Convolutional Neural Network 113 may be implemented as a deep learning model specialized for image analysis and may, for example, utilize multiple convolutional layers to analyze user-submitted skin images. The Transformer Network 114 may process product ingredient interactions and compatibility and may, in some implementations, utilize attention mechanisms to understand relationships between different skincare ingredients. The Decision Tree Model 115 may handle categorical features and decision-making processes and may, for example, process user preferences and environmental factors to generate routine recommendations.

In certain embodiments, the Data Layer 119 may include a Product Database 116, a User Database 117, and a Routine Database 118. The Product Database 116 may be implemented as a structured database containing information about skincare products and may, for example, include ingredients, usage instructions, and compatibility factors. The User Database 117 may store user-specific information and preferences and may, in some implementations, maintain historical data about user feedback and routine modifications. The Routine Database 118 may maintain personalized skincare routines and may, for example, track active routines, modification history, and product usage patterns.

In operation, according to some embodiments, user input received through the User Interface 104 may be processed by the Application Layer 102, where the Product Analysis Service 107 and ML Processing Service 108 may cooperate to analyze product compatibility and generate recommendations. The Routine Management Service 109 may coordinate with the Environmental Tracking Service 110 to adjust routines based on environmental factors. The Notification Service 111 may generate alerts and reminders based on routine schedules and product expiration dates, while the Budget Tracking Service 112 may monitor and report on routine-related expenses. The ML Model Layer 103 may provide analysis capabilities, with each model potentially specialized for specific types of data processing. The Data Layer 119 may maintain persistent storage of system data, potentially enabling optimization and personalization of user skincare routines.

In many embodiments, the present invention may include a user interface and associated logic flow that may include a login and signup page that assists with the intake of an end user to facilitate collection of high level information, a skin quiz set of pages that may include manual entry or image collection for the purposes of collecting information about an end user and their skin care and related health identification characteristics, one or more product screens that may include information correlated with a plurality of products that exist for skin care with associated data, information, reviews, ingredients, uses, and other associated information presented in a structured and digestible manner. In some embodiments, the product screens may include additional information including, without limitation, price, links for purchase, similar products, uses based on skin type, routine matching information, how such products may help certain individuals, as well as use instructions. In certain embodiments, the present invention may include a routine page that may assist with determining whether a product is compatible with a user’s skin type and skin care routine wherein the user could initiate the addition of certain products using an add to routine button. In some embodiments, the present invention may automatically add additional products into available slots of an end user’s skin care routine based on current products in an already existing routine.

In many embodiments, if a product is compatible with a user’s skin type but not the user’s routine, the present invention may be configured to indicate to the user why the product does not work with the user’s current routine for certain identified reasons, such as, for example, situations wherein there may be a conflict of ingredients between certain products or with a product a user may currently using in a routine or in many embodiments, when a product category is currently full.

In some embodiments, the present invention may be configured to enable a user to remove conflicting product to add in a new product or, for example, swap out a current product for a new product in a certain category.

In many embodiments, the present invention may be configured to enable a user view both AM and PM routines, as well as varying routines based off, for example, the day of the week. In some embodiments, there may be pre-configured routines optimized for skin care health factors that may be in the form of a plurality of steps in a daytime routine or nighttime routine, such as, for example, a cleanser, toner, moisturizer, serum, or SPF based product category. In many embodiments, the present invention may be configured to enable users to add in additional routine steps such as, for example, additional serums, oils, exfoliators, masks, peels, or spot treatments.

In some embodiments, within a routine there may be wait times displayed for products that may need a certain amount of time to reach effectiveness.

In many embodiments, the present invention may also include a vanity feature that may be configured to encompass all of the products that a user currently owns or uses in their current routine. In some embodiments the vanity feature may be configured to enable users to add preexisting catalogs of products to effectively categorize such products or modify active and inactive products that may be used in their skin care routine.

In some embodiments, the present invention may integrate features that may ease the often-cumbersome experience born out of an inundated market and the lack of easily accessible, tailored information. The system may be designed, in some embodiments, in such a way to enable a user to digest relevant information sources without having to search for or decipher whether such information may be truthful, accurate, or applicable for the situation and for the needs of a particular skin type or health factor. Additionally, in some embodiments, the present invention may be configured to predict certain needs of end users to provide benefits, such as, for example, rapid and customized feedback of products in a live real time manner based on user preferences as well as product ingredients among other product details. The present invention may, in some embodiments, include modules that help educate an end user of the system about products so they do not have to be an expert in skincare to understand the reason why a certain product may be better for a person’s particular needs. This is particularly significant in a landscape where product packaging and even brand websites often lack comprehensive information, compelling users to consult multiple sources to make an educated choice.

In some embodiments, the present invention may provide a solution for customized routine creation and real-time feedback as to how a potential new product may interact or fit within a user’s current skincare routine. In some embodiments, the invention may enable a user to quickly understand what products may work with the user’s specific skin type and target their concerns without the need for viewing any product packaging.

In some embodiments, the present invention may initiate user software flow by providing a skin quiz that may define a certain number of filters that may be applied to search results for users to narrow an overwhelming market of products that may or may not be right for the user’s specific needs or preferences. In some embodiments, there may be information input from users of the invention such as, for example, age categories, skin types, such as, for example, dry skin, combination skin, oily skin, and normal skin. In certain embodiments, the present invention may collect information relating to skin concerns of a certain individual, including, without limitation, issues such as breakouts, blemishes, skin dullness, dark spots, hyperpigmentation, dark under eye circles, dryness, enlarged pores, blackheads, free radical damage, fine lines, wrinkles, loss of firmness, redness, or uneven skin texture. The present invention may include an individual’s product or brand preferences, such as, for example, cruelty-free, gluten-free, vegan, eco-friendly brands, clean products only preference type, or, for example, an option to be shown all product types with or without preference. In many embodiments, the present invention may include features that, for example, may be based on a user's answers to a skin quiz, wherein the system may rate products on a percentage scale to determine whether a product is a match and assist end users of the system with choosing specific products.

In some embodiments, the present invention may be implemented through instructions stored on non-transitory computer-readable storage media, wherein such instructions may, when executed by one or more processors, cause a computer system to perform the methods and functionalities described herein.

In certain embodiments, the present invention may be structured to overcome issues involving individuals being overwhelmed from the inundation of products on the market and inability to easily find information so a user does not have to sift through an exhaustive number of products that would be impossible to sort through in a reasonable amount of time. In some embodiments, the present invention may provide efficient customized feedback tailored to the individual in a dynamic manner without having to be an expert in skin care product subject matter or development. In some embodiments, the present invention may solve the issues of consumers not being able to access information regarding certain products wherein even the product website does not properly disclose or keep information in a single place without having to search across the internet for certain pieces of information in a fragmented manner. For instance, some of the brand websites do not have all of the information that the present invention is able to structure and put in one place for a user to easily understand so they do not have to dive through a labyrinth of information in unstructured and fragmented websites, reviews, and unorganized site maps across multiple sources to make an attempt at an educated choice as to what the user should use.

In some embodiments the database components may include, for example, information relating to skin types such as oily, dry, sensitive, or combinations of such factors that may serve as baseline for product prediction and selection processes of the present invention. An additional factor that may be stored and used in the present invention may be age, as the skin's needs evolve over time, requiring different active ingredients and formulations to address concerns such as acne or signs of aging. Lifestyle choices, such as diet and stress levels, may also be included in some embodiments to influence skin condition and may be accounted for in a personalized skincare routine recommended by the present invention. Additionally, the present invention may be configured to include environmental factors, including, without limitation, climate and exposure to pollutants, which may play a significant role in skin health. UV exposure, for example, may necessitate products with added sun protection, which may be identified by the present invention based on a person providing such information or be based on location of an individual if such individual lives in an area that has high UV exposure. Furthermore, specific skin conditions, like eczema or rosacea, can be identified by the present invention to change the recommended products that should be used by a certain individual. Genetic predispositions may also be integrated into the present invention’s databases and prediction logic to offer insights into how one's skin might react to certain ingredients.

In some embodiments, the database components may be configured to balance optimal performance, scalability, and data integrity. Such database components may utilize SQL databases, such as, for example, SQLite or MySQL. Such database components may be serverless, self-contained database engines optimized for mobile and embedded platforms. In further embodiments, database components may store data and information in local files to assist with quick data retrieval and reduced resource usage. In such database components, some embodiments may also be configured to function in a mobile or web application environment or in a distributed form and may provide a robust set of features for complex queries and data manipulation. In some embodiments, the present invention may implement a microservices architecture with dedicated services for data ingestion, processing, and analysis. The data pipeline may, for example, handle continuous updates of product information, clinical studies, and user feedback through stream processing. In certain embodiments, a distributed cache layer may maintain frequently accessed data such as ingredient compatibility matrices and user profiles. The present invention may, in some embodiments, use probabilistic data structures such as Bloom filters to efficiently check product conflicts without exhaustive database queries.

In many embodiments, NoSQL databases may be integrated into the present invention to enable efficient, scalable, and flexible data storage and retrieval. In more embodiments, there may be document-based NoSQL databases, such as, for example, MongoDB, where such NoSQL databases may store unstructured or semi-structured data in, for example, a JSON or similar data structure or markup format. In certain embodiments use of a NoSQL databases may be configured to provide flexibility for the present invention to evolve rapidly and adapt to a changing code base. In some embodiments, the present invention may integrate key-value stores to enable high performance for scenarios requiring frequent read and write operations with simple data models. In further embodiments, graph databases may be integrated into the present invention to handle interconnected, relationship-heavy data such as the connections between a user’s skin care type, preferences, and health characteristics and the outputs of a sufficiently trained prediction model. In many embodiments, the present invention may utilize containerized deployment with automatic scaling based on user load. Database sharding may, for example, distribute user data across multiple nodes based on geographical location to enable low-latency access. In some embodiments, the prediction models may be deployed on GPU-enabled containers for parallel processing of multiple user requests. A message queue system may, in certain embodiments, handle asynchronous tasks such as routine optimization and notification delivery, wherein such system may ensure responsive user experience even under high load.

In certain embodiments, there may be a prediction or matching module for assessment of potential individual product issues and benefits to identify the best products to include in an individual’s skin care routine. For example, in many embodiments, statistical methodologies may be integrated into the invention to serve as an assessment mechanism to identify the efficacy and safety of a product for different skin types. In certain embodiments, the present invention may leverage statistics and data visualization techniques to identify correlations between a person’s skin type and other characteristics with certain products on the market and how the use or combination of products, individually or as a whole, may change the outcome of using such products. In some embodiments, machine learning algorithms may also be used to analyze a wealth of dermatological data, including user-generated feedback and clinical studies, to draw statistical inferences. For example, the present invention could use logistic regression or Bayesian models to calculate the probability of a product being effective for a specific skin type, such as oily or sensitive, based on variables such as, for example, ingredient concentration, pH level, or user reviews. At the same time, the present invention also employ’s hypothesis testing to discern whether the same product may result in adverse effects for other skin types, such as dry or combination skin types. By using a combination of descriptive and inferential statistics, the present invention may offer tailored product recommendations, enhancing user satisfaction while minimizing the risk of negative skin reactions. This data-driven approach empowers consumers to make informed choices without the need to conduct extensive data collection, research, and analysis to determine what products fit their specific needs. In many embodiments, the prediction module may employ learning modules that may, for example, associate gradient boosted decision trees with neural networks. For example, decision trees may handle categorical features such as skin type and product categories, while neural networks may process continuous variables including ingredient concentrations or pH levels. In some embodiments, this hybrid approach may enable both interpretable rules and complex pattern recognition. The present invention may, in some embodiments, maintain separate models for different prediction tasks, such as product compatibility, adverse reaction risk, and skin care routine optimization, wherein each model may be regularly retrained on anonymized user feedback data to improve accuracy.

In some embodiments artificial intelligence models may assist with the prediction or classifying of effects of skin care products in relation to specific skin characteristics. Utilizing machine learning algorithms, the present invention may, in some embodiments, analyze multidimensional data points to forecast the interaction between various skin care formulations and individual skin types. The primary inputs for the model may, for example, include high-resolution images, biochemical markers, and patient-reported metrics such as skin sensitivity levels or pre-existing conditions. Additionally, further embodiments may include ingredient compositions of skin care products as inputs that may be inputted as categorical or numerical variables. In some embodiments, the machine learning architecture may include decision tree models specifically configured for handling categorical features such as skin types, product categories, and user preferences.

In some embodiments, the present invention may implement a multi-modal deep learning architecture that may combine convolutional neural networks (CNNs) for image processing and transformer networks for analyzing ingredient interactions. The CNN component may, for example, process user-submitted skin images through multiple convolutional layers with max pooling to detect features such as texture, tone, and potential conditions. In certain embodiments, the transformer network may analyze ingredient lists as sequences, wherein self-attention mechanisms may identify complex interactions between components. Such networks may be trained on dermatological datasets and clinical studies to recognize patterns between skin characteristics and product efficacy.

In certain embodiments, such research and modeling techniques may be integrated into the present invention through, for example, a consumer mobile application, web application, or other software implementation, the artificial intelligence model predictive logic may be, for example hosted on a cloud server and be accessible via API calls. In certain embodiments, the present invention may obtain user consent and collect the requisite data by leveraging, for example, manual entry of skin care and other personal characteristics. In some embodiments, data and information collection may utilize built-in camera functionality for skin imaging to predict the type of skin care needs based on image classification. In many embodiments, once the pertinent information is collected from the end user of the present invention, it may transmit such data securely to a cloud or database stored model or prediction logic flow. In certain embodiments, the prediction logic or artificial intelligence engine may then process the inputs and return a classified or predicted outcome, which, in many embodiments, would be rendered for display on user interface, providing actionable insights into the compatibility between the chosen skin care product and the user's unique skin profile.

In many embodiments, the present invention may predict and warn of adverse effects in the same manner described above wherein the predictive logic or artificial intelligence model may provide a recommended combination of products for a user’s skin care routine, it may advise certain products or combinations that should be avoided to prevent adverse effects.

In some embodiments, the present invention may comprise a module for providing real-time routine fitting options that allow end users to scan product barcodes or take pictures of a product to identify information about the product and whether it may fit into their specific needs and routine. In certain embodiments, the present invention may identify a conflict with another product that a user may already have in their routine, or if such a user already owns a similar product. In addition, the present invention may automatically categorize a product into an AM or PM slot and on specific days of the week depending on the usage directions such product provides. In certain embodiments, the real-time routine fitting module may utilize computer vision and optical character recognition (OCR) to extract product information from images or barcodes. A low-latency inference pipeline may, in some embodiments, process this data through multiple stages that may include, for example, ingredient extraction and normalization, concentration analysis, and compatibility checking against existing routine products. In many embodiments, the system may maintain an in-memory cache of common product combinations to enable rapid response times for routine compatibility checks.

In some embodiments, the present invention may include features relating to budgeting and expense tracking wherein, for example, the system may track, summarize, render, and notify end users of costs and expenses relating to, for example, active, inactive, or incompatible products that may currently be stored in the vanity or other modules of the system for such users. Such features of the present invention may provide budget information upon initiation of a new product search that may, for example, display products within a budget set into the memory of the present system. Such features may include functionality, such as, for example, overall skin care budgets that could be set to daily, monthly, quarterly, annually, or any other budget time frame necessary for integration into a specific routine.

In certain embodiments, the present invention may also integrate a wish list module wherein certain products could be saved in memory when designated as a product that needs to be saved for later reference. End users of the system may be able to access the system through a user interface wherein they may be able to manually save such products or be presented certain products to be saved when the system predicts that such a product would be a match for the user’s needs. The present invention may also detect the geographical location of where the instance of the present invention is currently located and assess the proximity of certain products. Such features may include, for example, detection of certain stores that carry specific product inventory and, for example, whether there is stock of such product inventory on a real-time basis that may be indexed in real time for searching or used for sending alerts to the device wherein the system is currently integrated.

In some embodiment, the present invention may include new product release modules that may be based on answers provided by a user to the system through, for example, a skin quiz, wherein the system may be configured to provide alerts when a new product enters the market and, for example, may match a predetermined skin type, concern, or preference, saved in the system and relating to a specific user of the system. In some embodiments, the prediction module may utilize multiple machine learning model architectures working in parallel. For example, a convolutional neural network may analyze skin images, while a graph neural network may process ingredient interaction data, and a transformer network may handle textual product descriptions. The outputs of these models may be combined through an attention-based fusion mechanism to generate final product recommendations.

In some embodiments, the present invention may be configured to have a run through routines module wherein the system may enable a user to run through a user’s specific routines. In various embodiments, for example, such module features may include a start button and screen navigation features wherein following each screen would be a certain step of the user’s routine that may integrate product instructions and a timer that may indicate the wait times relating to a specific routine. If, for example, a user uses a product that requires a 10 min wait before the next product should be applied, the user may exit the system and still be sent a push notification when the system determines that it is time for the user to continue the routine.

In many embodiments, the present invention may be configured to include modules relating to tracking of specific information associated with products, routines, travel, or unique circumstances of a user. For example, some embodiments may include trackers relating to expiration dates of certain products that may be based on dates the product was opened, or for example, if there are product recalls from a provider that indicate that a product should no longer be used. Some embodiments may integrate a travel tracking module wherein the system may enable a user to modify their routine based on environmental factors such as, for example, weather conditions, skin type, user concerns, and various other preferences. In such embodiments, the present invention may be configured to determine desired or necessary changes to a skin care routine or product use based on weather conditions within a specific location in real time. In certain embodiments, the present invention may provide specific recommendations for routine modifications based on detected changes in a user's location, environmental conditions such as humidity and UV exposure, and personal factors including stress levels or hormonal changes.

In many embodiments, the present invention may include features that, for example, may be based on a user's answers to a skin quiz, wherein the system may rate products on a percentage scale to determine whether a product is a match and assist end users of the system with choosing specific products.

In some embodiments, the present invention may be configured with a prescription products module wherein users may save certain prescription product preferences within the system. In such embodiments, the system may include features wherein products may be screened or suggested based on conflicts with other products or complications such as allergies or potential adverse reactions to combination of certain ingredients in prescription products.

In many embodiments, the present invention may be configured with a custom products module wherein such module may provide the ability for a user to enter in product information to create a custom products for themselves that can be added to a routine and check for conflicts. In some embodiments, such modules may include the ability to discover and identify additional products that may be new or not generally commercially available wherein, for example, a user may discover a new product that is not currently in the system’s database and subsequently upload such product’s information for validation by the system. In such embodiments, once a product is validated, such products may then persist in the database and be accessed by the uploading user as well as all other users of the system. In such embodiments, the present invention may include features relating to customization of clean beauty preferences wherein a user may determine whether certain product ingredients are clean based on their own definition of clean products. Such customization features may enable a user to screen products based on ingredients, company, business practices, among other factors a user determines to be healthy, ethical, or otherwise.

In many embodiments, the present invention includes routine customization and automation modules that provide flexibility for the user to select when they want to use a product. For example, if the system’s default suggested use is to use a product in the morning or evening a user could also modify such use time frame to a time based on their current needs. Such customization features may provide the ability to, for example, change times, days, or frequency of product use in a routine. In some embodiments, the present invention may fully automate a routine for a user of the system. For example, the present invention may include a “generate routine” trigger action that would automatically create a routine for the user based on certain indicators, such as, for example, the products they have in their vanity, skin type, concerns, or preferences. Such automation module features may be configured to generate a best match routine for a user based on such indicators. Additionally, such module features may include the ability to “lock” certain products that a user would not want to be removed from a specific routine unlock products they would like swapped out by the routine generation features.

In certain embodiments, the present invention may be configured to have shopping modules that include features that may enable a user to see a list of retailers that carry certain products to determine specific purchasing information relating to products, such as, for example, if a product is cheaper at one location than another, a user could purchase from the desired location based on such information.

In certain embodiments, the present invention may implement a hierarchical product categorization system. Such system may, for example, classify products into primary categories such as "cleansers" or "moisturizers", secondary categories such as "oil-based" or "water-based", and tertiary categories based on key active ingredients. This hierarchical structure may enable efficient filtering and matching of products to user needs.

In some embodiments, the present invention may utilize a dynamic scoring system for product compatibility. Such system may assign weights to different factors including, for example:

Primary skin type match (weight: 0.4)

Secondary skin concerns match (weight: 0.3)

Ingredient compatibility (weight: 0.2)

Environmental factors (weight: 0.1)

In certain embodiments, the present invention may implement real-time ingredient conflict detection. For example, if a user selects a vitamin C serum, the system may automatically flag potential conflicts with products containing niacinamide in their routine. The system may, in some embodiments, suggest optimal timing gaps between potentially conflicting ingredients.

In many embodiments, the present invention may include an adaptive recommendation engine. Such engine may, for example, track seasonal changes in a user's location and automatically suggest routine modifications. For instance, the system may recommend increasing moisturizer concentration during winter months or adjusting SPF levels based on UV index forecasts. In some embodiments, the present invention may implement a multi-stage product matching pipeline that may include:

Initial filtering based on skin type and concerns

Ingredient compatibility analysis

pH level compatibility check

Climate suitability assessment

User preference matching

Budget consideration

Final ranking and recommendation generation

In certain embodiments, the present invention may utilize natural language processing to analyze product reviews and extract meaningful insights. Such analysis may, for example, identify patterns in user experiences with specific skin types, helping to refine product recommendations based on real-world usage data.

In some embodiments, the present invention may implement a dynamic routine optimization system that may adjust recommendations based on multiple factors including:

Time of year

Local humidity levels

Air quality index

User's stress levels

Sleep patterns

Hormonal cycles

Travel plans

Exercise routines

In many embodiments, the present invention may include a product efficacy tracking system. Such system may, for example, utilize computer vision to analyze user-submitted progress photos over time, measuring changes in skin texture, tone, and specific concerns to validate and refine product recommendations.

According to another aspect of the present invention, there is provided a non-transitory computer-readable medium including executable instructions that, when executed with one or more processors, cause the system or method for health product and routine management to perform any of the methods as described above. The method and system may be arranged to be performed by one or more processing components with any portions of software stored in an executable format on a computer readable medium. Thus, any portions of the method and system implemented as software may be stored in one or more non-transitory memory storage devices in an executable format to be executed by one or more processors. The computer readable medium may be non-transitory and may not include radio or other carrier waves. The computer readable medium could be, for example, a physical computer readable medium such as semiconductor memory or solid-state memory, magnetic tape, a removable computer diskette, a random access memory (RAM), a read-only memory (ROM), a rigid magnetic disc, and an optical disk, such as a CD-ROM, CD-R/W or DVD. The various methods described above may be implemented by a computer program product. The computer program product may include computer code arranged to instruct a computer to perform the functions of one or more of the various methods described above. The computer program and/or the code for performing such methods may be provided to an apparatus, such as a computer, on a computer readable medium or computer program product. For the computer program product, a transitory computer readable medium may include radio or other carrier waves.

The foregoing description of the preferred embodiment of the invention has been presented for the purposes of illustration and description. It is not intended to be exhaustive or to limit the invention to the precise form disclosed. Many modifications and variations are possible considering the above teaching. It is intended that the scope of the invention is not limited by this detailed description, but by the claims and the equivalents to the claims appended hereto.

Although a very narrow claim is presented herein, it should be recognized that the scope of this invention is much broader than presented by the claim. It is intended that broader claims will be submitted in an application that claims the benefit of priority from this application.

What is claimed is:

1. A computer-implemented method for managing a personalized skin care routine, the method comprising:
receiving user information related to at least one skin type, skin concern, product preference, and environmental factor;
accessing a database comprising information about a plurality of skin care products;
analyzing the user information and the skin care product information to identify a compatible product;
generating a personalized skin care routine comprising one or more of the compatible products; and
presenting the personalized skin care routine.

2. The method of claim 1, further comprising:
receiving feedback from the user regarding the personalized skin care routine; and
updating the personalized skin care routine based on the user feedback.

3. The method of claim 2, wherein analyzing the user information and the skin care product information comprises applying one or more machine learning models to predict compatibility between the user information and the skin care products.

4. The method of claim 3, wherein the one or more machine learning models comprise:
a convolutional neural network configured to analyze user skin images;
a transformer network configured to process ingredient sequence data to determine ingredient interactions and compatibility; and
a decision tree model configured to process categorical features comprising user preferences and environmental factors wherein the machine learning models are orchestrated by a machine learning processing service that routes different types of input data to appropriate models.

5. The method of claim 4, further comprising providing real-time feedback to the user regarding compatibility of a new product with the user's existing skin care routine.

6. The method of claim 5, wherein providing real-time feedback comprises:
receiving information identifying the new product;
analyzing the new product information in relation to the user's existing skin care routine; and
indicating whether the new product is compatible with the user's existing skin care routine.

7. The method of claim 6, further comprising tracking expiration dates of products in the user's skin care routine and providing notifications to the user regarding expired or soon-to-expire products.

8. The method of claim 7, further comprising providing recommendations for modifying the user's skin care routine based on changes in at least one of the user's location, environmental conditions, and personal factors.

9. The method of claim 8, wherein the computer-implemented method is implemented in a multi-layer architecture comprising:
a user interface layer comprising a user interface, a customization interface, and a notification center;
an application layer comprising a routine management service, a product analysis service, an ML processing service, an environmental tracking service, a notification service, and a budget tracking service;
a machine learning model layer comprising the one or more machine learning models; and
a data layer comprising a product database, a user database, and a routine database.

10. The method of claim 9, wherein analyzing the user information comprises:
routing image data to a convolutional neural network through an ML processing service;
routing product ingredient data to a transformer network for compatibility analysis;
routing categorical user preference data to a decision tree model;
aggregating outputs from the multiple machine learning models to generate a comprehensive compatibility assessment.

11. The method of claim 10, wherein the database comprises:
a product database storing product information and ingredient data;
a user database storing user preferences, feedback history, and environmental factors; and
a routine database storing active routines, modification history, and product usage patterns.

12. The method of claim 11, wherein providing recommendations based on environmental conditions comprises:
receiving real-time environmental data through an environmental tracking service;
analyzing the environmental data in conjunction with stored user sensitivity data;
generating routine modifications based on predicted environmental impacts; and
pushing notifications through a notification service when routine modifications are recommended.

13. The method of claim 12, comprising a non-transitory computer-readable medium including executable instructions that, when executed with one or more processors, cause a system for managing a personalized skin care routine to perform the methods therein.

14. A system for managing a personalized skin care routine, the system comprising:
one or more processors; and
a memory storing instructions that, when executed by the one or more processors, cause the system to:
receive user information related to at least one of a user's skin type, skin concerns, product preferences, and environmental factors;
access a database comprising information about a plurality of skin care products;
analyze the user information and the skin care product information to identify compatible products for the user;
generate a personalized skin care routine for the user comprising one or more of the compatible products; and
provide the personalized skin care routine to the user.

15. The system of claim 14, wherein the instructions further cause the system to:
receive feedback from the user regarding the personalized skin care routine; and
update the personalized skin care routine based on the user feedback.

16. The system of claim 15, wherein analyzing the user information and the skin care product information comprises applying one or more machine learning models to predict compatibility between the user information and the skin care products.

17. The system of claim 16, wherein the instructions further cause the system to provide real-time feedback to the user regarding compatibility of a new product with the user's existing skin care routine.

18. The system of claim 17, wherein the instructions further cause the system to provide recommendations for modifying the user's skin care routine based on changes in at least one of the user's location, environmental conditions, and personal factors.

19. The system of claim 18, comprising a non-transitory computer-readable medium including executable instructions that, when executed with one or more processors, cause the system for managing a personalized skin care routine to perform the methods provided therein.

20. A non-transitory computer-readable medium storing instructions that, when executed by one or more processors, cause a computer system to:
receive user information related to at least one of a user's skin type, skin concerns, product preferences, and environmental factors;
access a database comprising information about a plurality of skin care products;
analyze the user information and the skin care product information to identify compatible products for the user;
generate a personalized skin care routine for the user comprising one or more of the compatible products; and
provide the personalized skin care routine to the user.
